(19) United States
(12) Patent Application Publication
(10) Pub. No.: US 2011/0142793 A1
(43) Pub. Date: Jun. 16, 2011

(54) COMPOSITION FOR TREATING ARTICULAR CARTILAGE DEFECT, AND METHOD OF MANUFACTURE THEREOF

(75) Inventor: WING-PONG CHAN, Taipei City (TW)

(73) Assignee: TAIPEI MEDICAL UNIVERSITY, TAIPEI CITY (TW)

(21) Appl. No.: 12/825,287

(22) Filed: Jun. 28, 2010

(30) Foreign Application Priority Data
Dec. 11, 2009 (TW) 098142566

(51) Int. Cl. A61K 35/14; A61P 19/00; A61P 21/00; A61K 38/20; A61K 38/19

(52) U.S. Cl. 424/85.2; 424/93.72; 424/85.1

(57) ABSTRACT

The present invention provides a composition for treating an articular cartilage defect and a manufacturing method thereof. The composition comprises a cartilage fragment and platelet-rich fibrin (PRF), and is transplanted to a surface of the defective cartilage of a mammal in order to treat the articular cartilage defect. Wherein the cartilage fragment is acquired from the cartilage of the same mammal, and the PRF is acquired from the blood of the same mammal.

### FIELD OF THE INVENTION

The present invention relates to a composition, and a manufacturing method thereof; and more particularly, to a composition for treating an articular cartilage defect, and a method of manufacture thereof.

### BACKGROUND OF THE INVENTION

Articular cartilage, which covers each end of the long bones in vivo, is a white smooth structure. The structure of the articular cartilage is significantly different from the structure of the cortical bone beneath it. The articular cartilage does not have any blood vessels or neurons, so its nutrients are diffused from the surrounding joint fluids, and thus, the nutrients are provided slowly, resulting in relatively slow metabolism in the articular cartilage. Bone tissues have abundant blood vessels and nerve networks, such that their metabolism is very fast. Also, because a nerve system exists in bones, any impact or rubbing of two bones can be serious and painful. Because the articular cartilage covers the bones, the bones do not directly contact each other, so walking or intense movement does not cause discomfort.

Causes of articular cartilage defect include the following. Joints are used for a long time, causing the cartilage to be damaged. Mechanical injuries are caused by accidents, with the result that the cartilage is torn and breaks down. The cartilage is eroded because of diseases, such as gout, rheumatoid arthritis, osteoarthritis, and so on. Articular cartilage is known to have a poor capacity for healing after injury. Once the articular cartilage is damaged, it cannot repair itself and therefore is worn down gradually. In addition, if the bone under the articular cartilage is damaged, pain or swelling may occur, resulting in walking difficulty and affecting daily life.

Traditional repair operations for treating articular cartilage defects or degeneration must be performed via two surgical procedures. At the first surgical procedure, the cartilage injury is cut away, and parts of the chondrocytes with no injury are taken out for cell culture incubation in vitro. After incubation for as short as three weeks or up to few months, at the second surgical procedure the incubated chondrocytes are implanted into the injured cartilage in the patient. Therefore, the patient will be traumatized by two surgical operations, and the cartilage defects take a long time to be repaired. Furthermore, cell culture in vitro has many disadvantages, such as contamination.

### SUMMARY OF THE INVENTION

In view of the aforementioned drawbacks in existing skills or techniques, an object of the present invention is to provide a composition for treating an articular cartilage defect, and a method of manufacture thereof, so as to solve problems of contamination during incubation of chondrocytes and transplant rejection in vivo, and to achieve the efficiency of a shorter recovery time.

To achieve the above object, the composition for treating the articular cartilage defect comprises a cartilage fragment and platelet-rich fibrin (PRF), which are mixed with each other for treating at least one injury of the cartilage of a mammal. The cartilage fragment may be obtained from autologous cartilage in the same mammal, and the PRF may be obtained from autologous blood in the same mammal.

In addition, the method of manufacture of the composition for treating the articular cartilage comprises the following steps. A cartilage fragment obtained autologously from a mammal is ground into a powder. Platelet-rich fibrin (PRF) obtained autologously from the mammal is provided. The obtained powder and the PRF are mixed to obtain the composition.

The composition for treating the articular cartilage defect and the method of manufacture thereof according to the present invention provide one or more of the following advantages:

(1) When a defective cartilage is removed from a mammal at the first surgical operation, the composition according to the present invention is implanted into the injury site. Therefore, the articular cartilage detect is repaired by a single surgical operation, and there is no need to perform a second surgical operation on the mammal.

(2) Because the composition according to the present invention is obtained autologously from the mammal to be surgically operated on, the mammal does not have transplant rejection.

(3) The composition according to the present invention comprises platelet-rich fibrin (PRF). PRF can promote growth factors to activate normal immune responses, accelerate blood vessel regeneration, and at the same time induce aggregation and differentiation of cyclic adult stem cells and mesenchymal stem cells (MSCs) in vivo to speed up tissue regeneration.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a flowchart of a method of manufacture of a composition for treating an articular cartilage defect according to the present invention;

FIG. 2 shows H&E staining of the regenerated cartilage matrix with the composition according to the present invention; and

FIG. 3 is a bar chart showing results of a compressive stiffness test in regenerated cartilage.

### DETAILED DESCRIPTION OF THE PREFERRED EMBODIMENTS

The present invention will now be described with some preferred embodiments thereof with reference to the accompanying drawings. It is understood that the experimental data shown in the embodiments are provided only for easy interpretation of the technical means of the present invention and should in no means be considered as restriction of the present invention.

### Embodiment 1

### A Composition for Treating an Articular Cartilage Defect

The present invention provides a composition comprising a cartilage fragment and platelet-rich fibrin (PRF) for treating an articular cartilage defect. The cartilage fragment and the PRF are mixed for treating at least one cartilage defect in a mammal. The cartilage fragment may be obtained from autologous cartilage in the same mammal, and preferably, may be obtained from a part of a cartilage with no injury. The PRF may be obtained from autologous blood in the same mammal. The cartilage fragment is ground into a powder and mixed with the PRF for filling in at least one injury in the articular cartilage.

The PRF comprises many kinds of growth factors and cytokines. The growth factors may comprise a transforming growth factor, a platelet-derived growth factor, an epidermal growth factor, a vascular endothelial growth factor, or an insulin-like growth factor, or a combination thereof. The transforming growth factor, such as TGFβ-1, can promote cell growth and differentiation and regulate immune functions.

In addition, the cytokines may comprise interleukins, such as IL-6, IL-1β or IL-4, or tumor necrosis factors, such as TNF-α. Therefore, the composition according to the present invention has the cytokines needed for inflammatory responses and repair responses to promote normal immune responses in mammals.

### Embodiment 2

### Method of Manufacture of the Composition for Treating an Articular Cartilage Defect

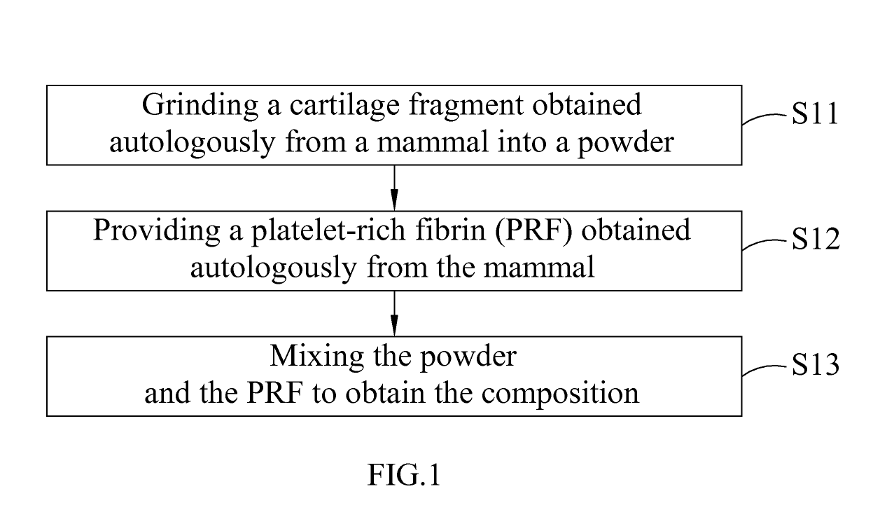

Please refer to FIG. 1, which is a flowchart of a method of manufacture of the composition for treating an articular cartilage defect according to the present invention. As shown, the steps include the following. In step S11, a cartilage fragment obtained autologously from a mammal is ground into a powder. In step S12, platelet-rich fibrin (PRF) obtained autologously from the said mammal is provided. In step S13, the powder and the PRF are mixed to obtain the composition. The cartilage fragment is obtained from a part of the cartilage with no injuries in the mammal: for example, the cartilage is obtained from normal cartilage adjacent to the injured cartilage.

The PRF is obtained from blood in the mammal, which is collected in a container which holds a separator polyester gel, and then centrifuged at a speed in the range of 1000 to 5000 rpm for 1 to 20 minutes. The centrifuge time is adjusted according to the centrifuge speed. Additionally, the blood may be centrifuged twice so as to obtain different concentrations of PRF.

In addition, PRF comprises many kinds of growth factors and cytokines. The growth factors may comprise a transforming growth factor, a platelet-derived growth factor, an epidermal growth factor, a vascular endothelial growth factor, or an insulin-like growth factor or a combination thereof. Further, the cytokines may comprise interleukins, such as IL-6, IL-1β or IL-4, or tumor necrosis factors, such as TNF-α. Therefore, the composition according to the present invention has the cytokines needed for inflammatory responses and repair responses to promote normal immune responses in mammals.

### Embodiment 3

### Supporting Tool (Kit) for Treating Articular Cartilage Defects

The supporting tool (kit) for treating articular cartilage defects comprises a centrifuge machine, a grinding device and a stirring device. The size of the supporting tool is convenient for placing in an operation room. The centrifuge machine is arranged for centrifuging blood to obtain platelet-rich fibrin (PRF), the centrifugation speed thereof is at least 1000 to 5000 rpm. The grinding device is arranged for grinding cartilage, and its components can be sterilized. The stirring device for mixing the PRF and the cartilage to obtain the composition, and the composition can be sent for sterilization.

### Embodiment 4

### Preferred Embodiments of the Present Invention

Preparation of PRF

Pigs are selected for use as laboratory animals in the present embodiment. Six ml blood is drawn from the pigs. The blood is collected in a container, such as a centrifuge tube, holding a separator polyester gel, and in the present embodiment, the centrifuge tube is used to contain the blood. The centrifuge tube containing the blood is centrifuged at a speed in the range of 1000 to 5000 rpm for 1 to 20 minutes, and preferably, also at 2500 to 3500 rpm for 8 to 12 minutes. The centrifuge time may be adjusted according to the centrifuge speed. After centrifugation, jelly-like PRF is obtained in the middle part of the centrifuge tube, and then sterile forceps are used to clip the jelly-like PRF out. All steps in the preparation of the PRF are performed under standard disinfection procedures. The 6 ml whole blood may yield 1-1.5 ml of PRF.

Experimental Design

Each pig has operations on the medial condylar surfaces of the distal femurs of both legs, such that the pig has two injuries, one in each leg, with diameters of 8 mm and depths of 5 mm, which imitate cartilage defects. The 16 pigs are divided into 4 groups: the PRF group, the P/C group, the CAR group, and the control group, according to the implantation substance, and thus, each group has 8 cartilage defects. Table 1 shows details of the implanted substances in each group.

TABLE 1
Each group and implantation substances
thereof in the present embodiment

| Group | Implantation substances |
|---|---|
| PRF group | Autologous PRF only |
| P/C group | Autologous PRF and an autologous cartilage fragment |
| CAR group | Autologous cartilage fragment only |
| Control group | No implanting of any substances; imitation of cartilage |
| | defects only |

The size of the autologous PRF is about 0.75 cm³, and the size of the autologous cartilage fragment is about 0.25 cm³. The autologous cartilage fragment may be further ground into a powder.

Six months after transplantation, the pigs in each group are sacrificed to analyze the degree of regeneration of the cartilage defect by means of a gross grading scale. The items in the gross grading scale include coverage, neo-cartilage color, defects in margins, and surface smoothness. Under the parameters of coverage, coverage of the regenerated cartilage over 75% on the medial condyle is defined as 4, and coverage ranges of 50-75%, 25-50%, less than 25% and without regenerated cartilages are defined as grades 3, 2, 1 and 0, respectively. The five degrees of neo-cartilage color are as follows: normal (white), 25% yellowish-brown, 50% yellowish-brown, 75% yellowish-brown and 100% yellowish-brown, defined as 4, 3, 2, 1 and 0, respectively. The margin defects are categorized as follows: no visible margin defect, 25% margin visibly defective, 50% margin visibly defective, 75% margin visibly defective and entire margin visibly defective are defined as 4, 3, 2, 1 and 0, respectively. Finally, the five degrees of surface smoothness are: smooth/level with normal surface, smooth but raised, 20-50% irregular, 50-75% irregular, and more than 75% irregular, defined as 4, 3, 2, 1, and 0, respectively. Therefore, the higher the score of the gross grading scale, the better the regeneration and recovery.

For the coverage of regenerated cartilage, the results indicate that the amount of regenerated cartilage in the control group is obviously inadequate. For example, the coverage of the regenerated cartilage in five cartilage defects is less than 50%, and one cartilage defect is still hollow without regenerated cartilage. Compared with the control group, in the PRF group, the coverage of regenerated cartilage in the six cartilage defects reaches more than 75%. In the CAR group, the coverage in four cartilage defects reaches 50%, and the coverage in two cartilage defects reaches only 25%. In the P/C group, the coverage of regenerated cartilage in all cartilage defects reaches more than 75%. In the neo-cartilage color analysis, the color of the regenerated cartilage in the PRF group and the CAR group range between white and red. But the color of the regenerated cartilage in the P/C group is white, and the surface of the regenerated cartilage is smooth, without damage in the margins. Table 2 shows the results from the gross grading scale.

TABLE 2
The results of the gross grading scale in
each group for the present embodiment

| | Values are mean ± standard deviation. | | | |
|---|---|---|---|---|
| | | Neo-cartilage | Defective | Surface |
| Group | Coverage | color | margins | smoothness |
| PRF group | 2.75 ± 0.31 | 1.97 ± 0.35 | 1.90 ± 0.24 | 1.53 ± 0.33 |
| P/C group | 3.56 ± 0.28 | 2.88 ± 0.31 | 2.88 ± 0.27 | 2.90 ± 0.24 |
| CAR group | 2.93 ± 0.28 | 2.34 ± 0.33 | 2.31 ± 0.33 | 2.50 ± 0.31 |
| Control | 1.59 ± 0.27 | 1.78 ± 0.22 | 1.13 ± 0.26 | 0.78 ± 0.27 |
| group | | | | |

The microstructure of the regenerated cartilage is analyzed by a histological scaling assessment. The histological scaling assessment of regenerated cartilage has six parameters-surface, matrix, cell distribution, cell population, subchondral bone and cartilage mineralization. The surface of regenerated cartilage being continuous and smooth is defined as 3, and the surface of regenerated cartilages being crude is defined as 0. For the matrix of regenerated cartilage, hyaline, hyaline/fibrocartilage, fibrocartilage and fibrous tissue are defined as 3, 2, 1 and 0, respectively. For the cell distribution of regenerated cartilage, columnar, columnar-cluster, cluster and individual cells/disorganized are defined as 3, 2, 1 and 0, respectively. For the cell population of regenerated cartilage, predominantly viable, partially viable and less than 10% viable are defined as 3, 1 and 0, respectively. For subchondral bone, normal, increased remodeling, bone necrosis/granulation tissue, detached/fracture/callus at base are defined as 3, 2, 1 and 0, respectively. Finally, for mineralization of the regenerated cartilage, normal and abnormal/inappropriate are defined as 3 and 0, respectively. When the calculated score is higher, the microstructure of the regenerated cartilage is more complete. Table 3 shows the results of the histological scaling assessment.

TABLE 3
The results of the histological scaling assessment
in each group for the present embodiment

| | Values are mean ± standard deviation. | | |
|---|---|---|---|
| Group | Surface | Matrix | Cell distribution |
| PRF group | 2.79 ± 0.27 | 1.14 ± 0.28 | 1.14 ± 0.28 |
| P/C group | 2.86 ± 0.29 | 2.18 ± 0.24 | 2.36 ± 0.32 |
| CAR group | 2.79 ± 0.30 | 1.79 ± 0.27 | 1.82 ± 0.19 |
| Control group | 1.00 ± 0.29 | 0.96 ± 0.22 | 0.64 ± 0.20 |
| | Values are mean ± standard deviation. | | |
| | | | Cartilage |
| Group | Cell population | Subchondral bone | mineralization |
| PRF group | 2.25 ± 0.29 | 2.00 ± 0.20 | 1.50 ± 0.20 |
| P/C group | 2.68 ± 0.35 | 2.43 ± 0.28 | 2.14 ± 0.31 |
| CAR group | 2.32 ± 0.35 | 2.14 ± 0.24 | 1.68 ± 0.35 |
| Control group | 2.21 ± 0.27 | 1.64 ± 0.20 | 0.68 ±0.19 |

To sum up the results, the scores for the matrix, the cell distribution and cartilage mineralization of the regenerated cartilages in the P/C group are significantly higher than those in the other three groups. Therefore, the microstructure of the regenerated cartilage in the P/C group is more complete than that in the other three groups.

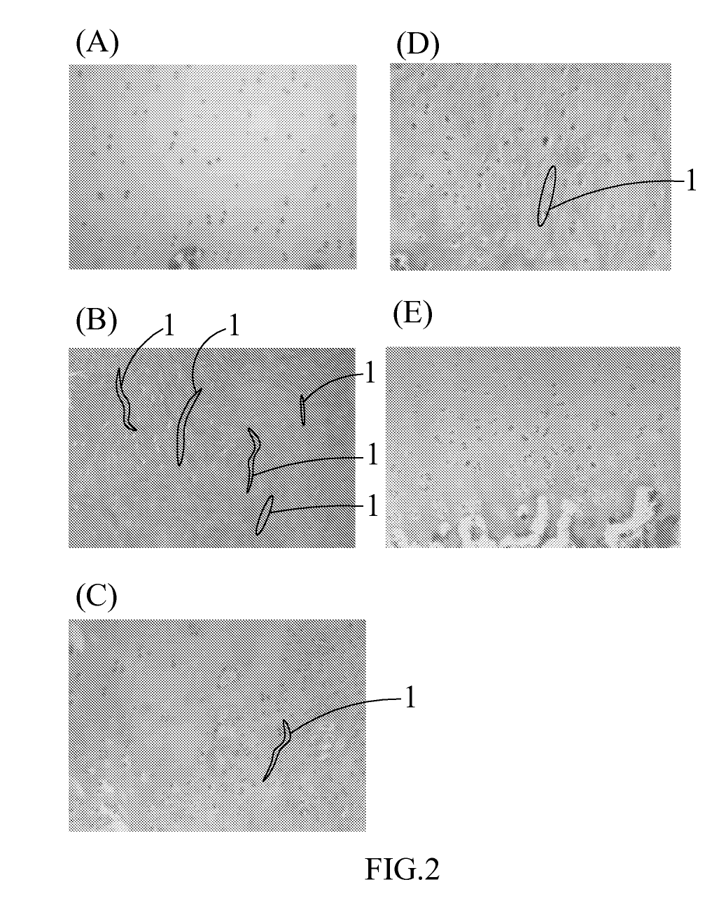

Please refer to FIG. 2, that is, H&E staining pictures of the regenerated cartilage matrix with the composition according to the present invention. As shown, sections (A), (B), (C), (D) and (E) in FIG. 2, respectively, represent non-damaged cartilage, the control group, the PRF group, the CAR group and the P/C group. Most of the matrix of the regenerated cartilage in the PRF group and the CAR group is integrated with hyaline cartilage and fibrous cartilage. The fibrous cartilage is usually converted to hard bone by ossification. The amount of fibrous cartilage in the P/C group is less, and the section of the P/C group is similar to the section of the non-damaged cartilage.

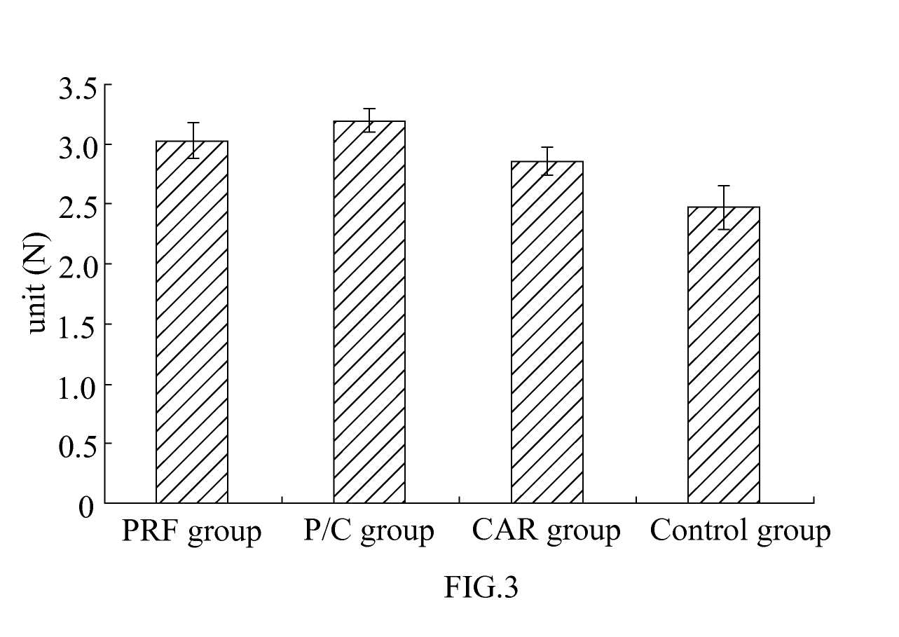

Please refer to FIG. 3, that is, a bar chart showing results of a compressive stiffness test on regenerated cartilage. As shown, the Newton (N) value in the P/C group is higher than in the other three groups. The major function of cartilage is to prevent contact of two hard bones. Therefore, the higher the Newton value in the compressive stiffness test, the better the neo-cartilage function.

Accumulations of sulphate-glycosaminoglycans (s-GSGs) are revealed and analyzed by Alcian blue staining. In normal cartilage, s-GSGs accumulate in the extra-chondrocyte matrix, so that it is presented as a uniform blue color. In the PRF group and the CAR group, the staining results range between those of the repaired cartilage and the non-damaged cartilage. However, the staining results in the P/C group and normal cartilage are similar, indicating that the repair efficacy in the P/C group is better. Furthermore, accumulation of proteoglycans in extra-chondrocytes are revealed and analyzed by safranin O. The results show the color of the regenerated cartilage in the P/C group is stronger than that in the PRF group and the CAR group, and is similar to that of the normal cartilage.

In summary, when the composition according to the present invention is used to treat an articular cartilage defect in a mammal (in the P/C group), the repair efficiency is higher than that in the other groups. When a defective cartilage is removed by a surgical operation, the composition is implanted into the injury site at the same time, thereby repairing the cartilage defect. Thus, there is no need to perform a second surgical operation. The composition is obtained autologously from a mammal, thereby obviating transplant rejection. The composition comprises PRF, and PRF comprises growth factors to activate normal immune responses. The growth factors can accelerate blood vessel regeneration, and at the same time induce aggregation and differentiation of cyclic adult stem cells and mesenchymal stem cells (MSCs) in vivo. In addition, because the composition comprises an autologous cartilage fragment, the autologous cartilage fragment can act as a scaffold to speed up tissue regeneration.

The present invention has been described with some preferred embodiments thereof, and it is understood that many changes and modifications in the described embodiments can be carried out without departing from the scope and the spirit of the invention that is intended to be limited only by the appended claims.

What is claimed is:

1. A composition for treating an articular cartilage defect, comprising:
a cartilage fragment obtained from autologous cartilage in a mammal; and
platelet-rich fibrin (PRF) obtained from autologous blood in the mammal, and mixed with the cartilage fragment.

2. The composition for treating the articular cartilage defect as claimed in claim 1, wherein the mammal has at least one injury of the cartilage.

3. The composition for treating the articular cartilage defect as claimed in claim 2, wherein the cartilage fragment is obtained from a part of the cartilage with no injury in the mammal.

4. The composition for treating the articular cartilage defect as claimed in claim 3, wherein the cartilage fragment is ground into a powder form and mixed with the PRF for filling in the at least one injury of the cartilage.

5. The composition for treating the articular cartilage defect as claimed in claim 1, wherein the PRF comprises a growth factor.

6. The composition for treating the articular cartilage defect as claimed in claim 5, wherein the growth factor comprises a transforming growth factor, a platelet-derived growth factor, an epidermal growth factor, a vascular endothelial growth factor, or an insulin-like growth factor, or a combination thereof.

7. The composition for treating the articular cartilage defect as claimed in claim 1, wherein the PRF further comprises a cytokine.

8. The composition for treating the articular cartilage defect as claimed in claim 7, wherein the cytokine comprises an interleukin or a tumor necrosis factor.

9. A method of manufacture of a composition for treating an articular cartilage defect, comprising the following steps:
grinding a cartilage fragment obtained autologously from a mammal into a powder;
providing platelet-rich fibrin (PRF) obtained autologously from the mammal; and
mixing the powder and the PRF to obtain the composition.

10. The method of manufacture of the composition for treating the articular cartilage defect as claimed in claim 9, wherein the cartilage fragment is obtained from undamaged cartilage in the mammal.

11. The method of manufacture of the composition for treating the articular cartilage defect as claimed in claim 9, wherein the PRF is obtained from blood after centrifugation.

12. The method of manufacture of the composition for treating the articular cartilage defect as claimed in claim 11, wherein the blood is collected in a container which holds a separator polyester gel.

13. The method of manufacture of the composition for treating the articular cartilage defect as claimed in claim 12, wherein a rotational speed of the centrifugation ranges between 1000 and 1500 rpm.

14. The method of manufacture of the composition for treating the articular cartilage defect as claimed in claim 13, wherein a rotational time of the centrifugation ranges between 1 and 20 minutes.

15. The method of manufacture of the composition for treating the articular cartilage defect as claimed in claim 9, wherein the PRF comprises a growth factor.

16. The method of manufacture of the composition for treating the articular cartilage defect as claimed in claim 15, wherein the growth factor comprises a transforming growth factor, a platelet-derived growth factor, an epidermal growth factor, a vascular endothelial growth factor, or an insulin-like growth factor, or a combination thereof.

17. The method of manufacture of the composition for treating the articular cartilage defect as claimed in claim 9, wherein the PRF further comprises a cytokine.

18. The method of manufacture of the composition for treating the articular cartilage defect as claimed in claim 17, wherein the cytokine comprises an interleukin or a tumor necrosis factor.
